(19) United States
(12) Patent Application Publication
(10) Pub. No.: US 2010/0076340 A1
(43) Pub. Date: Mar. 25, 2010

(54) DEVICE AND METHOD TO EXTRACT MATERIAL OF A MATERIAL RESERVOIR

(76) Inventors: Gerald Eckstein, Muenchen (DE); Karsten Hiltawsky, Schwerte (DE); Heinz-Ingo Schneider, Baldham (DE); Daniel Sickert, Muenchen (DE)

Correspondence Address: SCHIFF HARDIN, LLP;PATENT DEPARTMENT, 233 S. Wacker Drive-Suite 6600, CHICAGO, IL, 60606-6473, US

(21) Appl. No.: 12/566,009

(22) Filed: Sep. 24, 2009

(30) Foreign Application Priority Data
Sep. 25, 2008 (DE) 10 2008 048 866.6

(51) Int. Cl. A61B 10/02; A61B 10/00

(52) U.S. Cl. 600/562; 600/573

(57) ABSTRACT

A device to extract material of a material reservoir has at least one guide tube with internal space and with an inner wall surrounding the internal space; at least one sled that can implement a sled translation movement in the inner space along a longitudinal alignment of the guide tube; and at least one collection unit with collection surface to collect the material. The collection unit and the sled are coupled to one another such that the sled translation movement of the sled leads to a collection translation movement of the collection surface of the collection unit, and such that the collection surface can be inserted into the material reservoir and be withdrawn from the material reservoir. A rotation mechanism that is operated manually or automatically can be provided with which a wire, which acts as a collection device, can be displaced in vivo over a length of 1 mm to up to 2 cm within a venous cannula. The invention is used in medical technology, for example, to collect cells from a reservoir in the form of a bodily fluid.

### BACKGROUND OF THE INVENTION

1. Field of the Invention

The present invention concerns a device and a method to extract material of a material reservoir.

2. Description of the Prior Art

To collect sample material in the bloodstream as a material reservoir, collection devices in the form of thin wires are inserted into cannula situated in a blood vessel. The material accumulates at the wire tip of the thin wire. After the collection of the material, the entire wire must be drawn through the entire cannula. The wire contacts an external wall of the cannula tube, and an abrasive stress on the wire surface occurs and the collected material may be stripped off or altered.

### SUMMARY OF THE INVENTION

An object of the present invention to provide a method and device with which the aforementioned abrasive stress can be avoided.

This object is achieved in accordance with the invention by a device to extract material of a material reservoir, the device having at least one guide tube with an internal space and with an inner wall that defines the internal space; and at least one sled that can implement a sled translation movement in the inner space along a longitudinal alignment of the guide tube; and at least one collection unit with a collection surface to collect the material, wherein the collection unit and the sled are coupled to one another to cause the sled translation movement to produce a collection translation movement of the collection surface of the collection unit, that causes the collection surface to be inserted into the material reservoir and be extracted from the material reservoir.

The above object also is achieved in accordance with the invention by a method for the extraction of material of a material reservoir using a device as described above, the method including the method step of uniting the collection surface of the collection device for the execution of the sled translation movement of the sled, to enable an accumulation of the material of the material reservoir on the collection surface as a result of such uniting.

The underlying basis of the invention is to ensure a defined insertion of the collection surface into the material reservoir with the aid of the guide tube and the sled. It is assured that abrasive stresses of the collection surface (for example by the collection surface uncontrollably reaching a cannula wall of the cannula) do not occur either upon insertion of the collection surface into the material reservoir or upon removal of the collection surface from the material reservoir.

In an embodiment, the inner wall of the guide tube has a guide bearing aligned along the longitudinal alignment of the guide tube to guide a dog connected with the sled. The guide bearing is a groove into which the dog (pin) of the sled protrudes.

In a further embodiment, the guide bearing essentially runs parallel to the longitudinal axis of the guide tube. In this context, “essentially” means that deviations of up to 10° from an exact parallel alignment can occur.

In another embodiment, the guide bearing is tilted against the longitudinal alignment of the guide tube. The guide bearing and the longitudinal alignment are arranged at an angle to one another. An inclination or an angle of inclination results. Given a fixed (thus stationary) guide tube, the guidance of the dog in the guide bearing in addition to the sled translation movement leads to a sled rotation movement. If the collection unit with the collection surface and the sled are firmly connected with one another, a collection rotation movement results from the sled rotation movement.

A preferred solution for the described device is as follows: the guide tube is designed as a hollow cylinder and the sled as a piston. The piston can be moved positively in the hollow cylinder. The piston and hollow cylinder form a positive fit.

In another embodiment, a guide rail in the guide tube is provided to guide the sled is present in the inner space of the guide tube, such that the sled can essentially implement only the sled translation movement. The movement of the sled is decoupled from possible movement of the guide tube. This solution ensures that only the sled translation movement occurs. If the collection unit is firmly connected with the sled, only a collection translation movement can occur. The collection surface does not rotate. An additionally reduced abrasive stress of the collection surface results.

In an embodiment, a lifting device is provided to adjust a sled stroke of the sled along the longitudinal alignment of the guide tube. The lifting device has an actuator in the form of a motor, for example. By activating the actuator, the position (of the sled stroke) in the inner space of the guide tube is adjusted.

The lifting device can include the guide tube with the guide bearing. The guide tube is executed so that it can be rotated, such that a rotation of the guide tube leads to the sled translation movement. For example, this is possible when the sled can, as described above, perform the sled translation movement in a guide rail firmly connected with the guide tube. A simple rotation mechanism results in which the rotation of the guide tube and the sled translation movement are decoupled. The sled stroke is set by the rotation. If the collection surface and the sled are firmly connected with one another, no collection rotation movement occurs but rather only the collection translation movement. A collection stroke of the collection surface, and therefore a projection of the collection surface into the material reservoir, can easily be adjusted.

In a further embodiment, the guide bearing comprises at least one recess of the guide bearing to catch the dog. The sled stroke can be adjusted in stages via the catching of the dog in the recess. For catching it is advantageous if the dog can be flexed.

For the collection of the sample, the collection surface is correspondingly designed. In a particular embodiment, the collection surface is formed by a coating of the collection unit. The device can be modified in this way for the most varied fields of application. Which material accumulates on the surface is established solely by the coating. The material can be an inorganic material or an organic material. In particular, biological materials are conceivable in the form of cells or proteins.

In another embodiment, the collection unit has a wire. The collection surface is formed by a wire surface of the wire. For example, the collection surface is located at a wire tip of the wire. The wire tip is guided into the material reservoir with the aid of the device.

In order to further reduce the abrasive stress on the wire surface or, respectively, the collection surface, it is particularly advantageous to additionally provide a protective sleeve. The wire or the wire surface is inserted from the protective sleeve into the material reservoir or, respectively, is driven from the material reservoir into the protective sleeve. The protective sleeve is, for example, a cannula. The protective sleeve is thereby positively connected with the guide type, for example. A correspondingly adapted adapter is preferred for this. In the case of a material reservoir in the form of blood, an adapter is in particular used in the form of a Luer Lock connection.

The device can be used to extract material from a material reservoir in the most varied fields. A quality of the material reservoir can be checked via the extraction, for example. The quality depends on the composition of the material reservoir, for example. Aging processes of the material reservoir can be detected via decay products in said material reservoir, for example. Impurities can likewise be detected with the aid of the device. The evidence is arrived at particularly simple if the collection surface is designed as a component of a corresponding sensor.

Any fluids (liquids, gases, mixtures of gases and liquids) can be considered as material reservoirs. The field of application thus ranges from comestibles (for example water) to organic solvents and to lubricants and oils (for example motor oil). In particular a material reservoir of an organism is used as a material reservoir. The material reservoir of an organism is, for example, likewise a fluid in the form of a bodily fluid of the organism. The bodily fluid is, for example, blood, urine, intracellular fluid etc. With regard to an organism, the material reservoir can also be an accumulation of cells from which material should be extracted, for example via a puncture. In this case, the material reservoir would not be of a fluid nature.

The material to be collected can consist merely of small molecules. In particular, materials in the form of large, complex biological systems (for example in the form of proteins or cells are conceivable.

The following advantages of the invention should be emphasized:

With the invention it is possible to collect and to extract material on a collection surface of a collection unit without significant abrasive stress.

Via the proposed rotation mechanism, the collection surface (for example in the form of a wire) can be displaced by a defined length. According to a particular embodiment, the collection surface thereby does not rotate, which leads to an additionally reduced abrasive stress of the collection surface.

The collection surface in the form of a wire surface formed by a wire tip of a wire does not need to be drawn completely through a corresponding cannula.

### BRIEF DESCRIPTION OF THE DRAWINGS

FIG. 1 shows an embodiment of a device to extract material of a material reservoir in perspective representation, in accordance with the invention.

FIG. 2 shows a section of the device for extraction of material in a perspective representation.

FIG. 3 shows an exploded diagram of the device for extraction of material of a material reservoir.

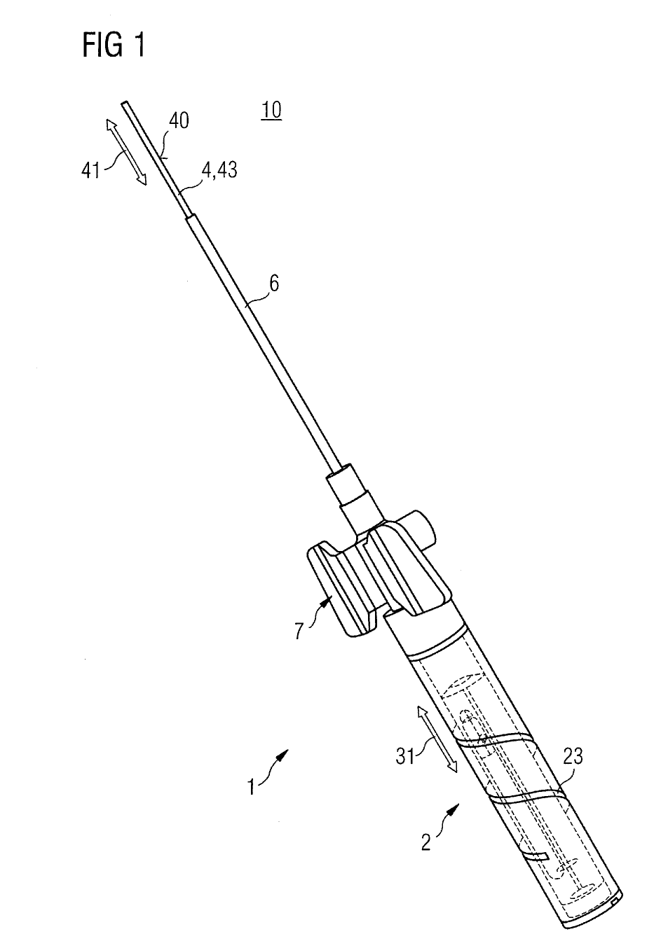

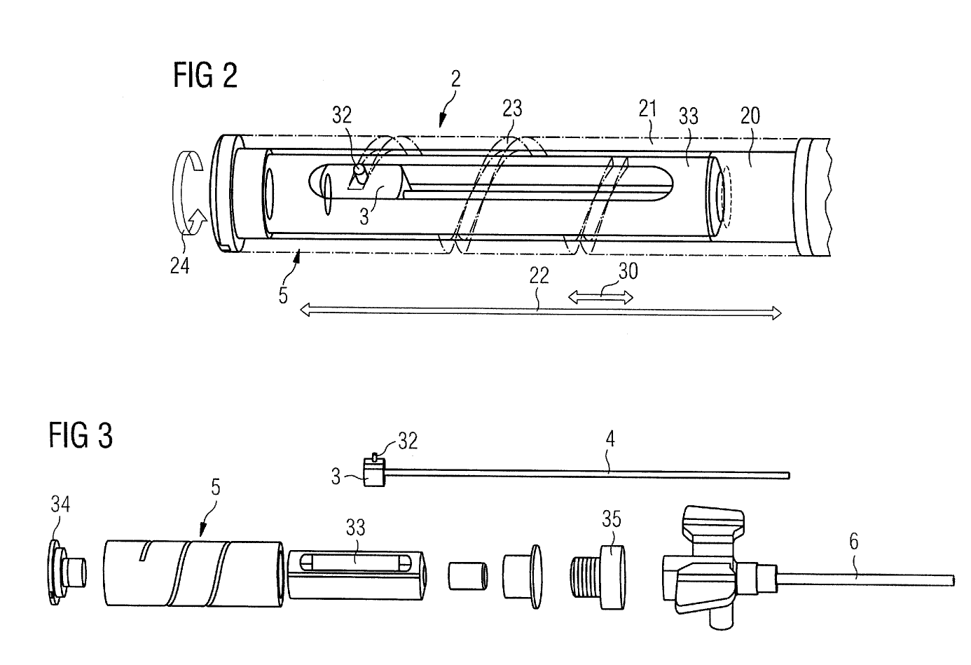

### DESCRIPTION OF THE PREFERRED EMBODIMENTS

The device 1 to extract material of a material reservoir 10 has a guide tube 2 with inner space 20 and with an inner wall 21 bounding the inner space. Inside the inner space of the guide tube, a sled 3 is arranged such that it can implement a sled translation movement 30 along a longitudinal alignment 22 of the guide tube.

Moreover, a collection unit 4 with collection surface 40 to collect the material is provided. The collection unit and the sled are coupled to one another such that the sled translation movement of the sled leads to a collection translation movement 41 of the collection surface of the collection unit, and such that the collection surface can be inserted into the reservoir and be withdrawn from the reservoir. The collection unit is [sic] by a wire and the collection surface by a wire surface at the end of the wire.

The inner wall of the guide tube possesses a guide bearing (groove) 23 aligned along the longitudinal alignment of the guide tube to guide a dog 32 connected with the sled. The guide bearing is tilted counter to the longitudinal alignment of the guide tube.

A guide rail 33 to guide the sled is present in the inner space of the guide tube, such that the sled can essentially implement only the sled translation movement. A sled rotation movement is not initiated by the sled translation movement. The sled stroke 31 of the sled ensues within the guide rail. For a firm connection of the guide rail with the guide tube, the guide rail is fixed in the guide tube with the bolts 34 and 35.

To adjust the sled stroke of the sled along the longitudinal alignment of the guide tube, a stroke device 5 is present. The stroke device possesses the guide tube with the guide bearing. The guide tube is thereby executed so that it rotates, such that a rotation 24 of the guide tube leads to the sled translation movement.

According to further embodiments (not shown), the guide rail possesses recesses in which the dog can catch. For this the dog can be flexed. As an alternative to this, the rail is borne elastically. For this the bolts 34 and 35 has resilient (spring) elements (not shown).

According to the exemplary embodiment, the material reservoir is blood that runs in a vein. The wire can be moved linearly within a protective sleeve 6 in the form of a vein cannula that is positively connected with a Luer Lock connection 7. A maximum stroke of the wire is dimensioned so that the free wire end with the collection surface possesses a projecting length (defined for the application) relative to the vein cannula. This projecting length with which the wire can protrude into the vein is up to 30 mm. Given a minimal stroke, the wire tip is submerged in the vein cannula. The wire tip disappears into the vein cannula. Due to the rotation mechanism, the wire can be displaced over a length of 1 mm to up to 2 mm within the vein cannula.

Although modifications and changes may be suggested by those skilled in the art, it is the intention of the inventors to embody within the patent warranted hereon all changes and modifications as reasonably and properly come within the scope of their contribution to the art.

We claim as our invention:

1. A device to extract material of a material reservoir, comprising:
at least one guide tube with an internal space and with an inner wall surrounding the internal space;
at least one sled that implements a sled translation movement in the inner space along a longitudinal alignment of the guide tube;
at least one collection unit having a collection surface to collect material; and
the collection unit and the sled being coupled to one another to cause the sled translation movement of the sled to produce a collection translation movement of the collection surface of the collection unit, to insert the collection surface into the material reservoir to withdraw the collection surface from the material reservoir.

2. A device as claimed in claim 1 wherein the inner wall of the guide tube has a guide bearing aligned along the longitudinal alignment of the guide tube to guide a dog connected with the sled.

3. A device as claimed in claim 2 wherein the guide bearing runs essentially parallel to the longitudinal direction of the guide tube.

4. A device as claimed in claim 2 wherein the guide bearing is tilted counter to the longitudinal alignment of the guide tube.

5. A device as claimed in claim 2 comprising a guide rail that guides the sled in the inner space of the guide tube, allowing the sled to implement essentially only the sled translation movement.

6. A device as claimed in claim 1 comprising a stroke device that adjusts the sled stroke of the sled along the longitudinal alignment of the guide tube.

7. A device as claimed in claim 6 wherein the stroke device includes the guide tube with a guide bearing, and the guide tube is configured to rotate, with a rotation of the guide tube producing the sled translation movement.

8. A device as claimed in claim 7 wherein the guide bearing comprises at least one recess of the guide bearing to receive a dog on the sled.

9. A device as claimed in claim 1 wherein the collection surface is formed by a coating of the collection unit.

10. A device as claimed in claim 1 wherein the collection unit comprises a wire, and the collection surface is formed by a wire surface of the wire.

11. A device as claimed in claim 1 comprising a protective sleeve that accommodates the collection unit.

12. A device as claimed in claim 11 wherein the protective sleeve is formed by a cannula.

13. A method for extracting material from a material reservoir, comprising the steps of:
providing at least one sled in an interior space of a guide tube surrounded by an inner wall;
executing a longitudinal movement of the sled in the inner space along a longitudinal direction of the guide tube;
coupling at least one collection unit, having a collection surface configured to collect material, to said sled to cause the translation movement of the sled to produce a translation movement of the collection surface of the collection unit; and
operating the coupled sled and collection unit to insert the collection surface into the material reservoir and to withdraw the collection surface from the material reservoir.

14. A method as claimed in claim 13 comprising collecting fluid from said material reservoir on said collection surface.

15. A method as claimed in claim 13 comprising inserting said collection surface into a material reservoir of an organism in vivo.

16. A method as claimed in claim 15 comprising collecting biological material in vivo on said collection surface.
